(19) United States
(12) Patent Application Publication
(10) Pub. No.: US 2016/0030325 A1
(43) Pub. Date: Feb. 4, 2016

(54) Method and Compositions for Treating Dermal Papilla Cells Associated with Keratin Fibers

(71) Applicant: ELC Management LLC, Melville, NY (US)

(72) Inventors: Jaimie Jerome, Setauket, NY (US); Daniel B. Yarosh, Merrick, NY (US); Thomas Mammome, Farmingdale, NY (US)

(21) Appl. No.: 14/448,109

(22) Filed: Jul. 31, 2014

(51) Int. Cl. A61K 8/66; A61Q 5/02; A61Q 5/00; C12N 9/88; A61K 8/99; A61Q 1/00

(52) U.S. Cl. CPC A61K 8/66; A61K 8/99; A61Q 1/00; A61Q 5/002; C12N 9/88; A61Q 5/02; A61K 2800/10; C12Y 402/99018

(57) ABSTRACT

A composition for topical application to dermal papilla cells associated with keratin fibers from scalp, eyelashes or eyebrows comprising at least one OGG1 DNA repair enzyme, and method for treating dermal papilla cells associated with keratin fibers to inhibit oxidative damage, stimulate melanin synthesis, and reduce apoptosis.

### TECHNICAL FIELD

The invention is in the field of compositions and ingredients for treating dermal papilla cells to inhibit oxidative damage which in turn promotes hair growth, reduces hair graying, and promotes strong and healthy hair.

### BACKGROUND OF THE INVENTION

The dermal papilla cells are critical to the growth of hair, such as eyelashes, eyebrows, or head hair. These cells are found at the bottom of the hair follicle. As hair grows it cycles through three distinct phases: anagen, catagen, and telogen. All three phases have specific characteristics that determine the length of hair. In addition, all three phases occur simultaneously; e.g. some hairs may be in the anagen phase, some hairs in the catagen phase, and other hairs in the telogen phase. The anagen phase is the growth phase. It begins in the dermal papilla cells and can last from two to six years. The catagen phase, also known as the transitional phase, allows the follicle to renew itself. This period lasts about two weeks. The follicle disintegrates and the hair shaft is cut off from its blood supply which causes the hair shaft to be pushed upward. During the telogen, or the resting phase, the follicle remains dormant from one to six months. The follicle eventually begins to grow again causing the hair to shed.

It is known that dermal papilla cells are affected by inflammation, environmental toxins, pollution, and simple stress. We have all heard stories about individuals who lose their hair overnight due to some stressful incident in their life, or others with hair that has very quickly turned gray due to physical or psychological trauma.

Hydrogen peroxide is a strong oxidizer which is generated in the form of reactive oxygen species. It is naturally produced in vivo as a result of metabolism and converted into water and oxygen via catalase peroxidase. Normal healthy levels of hydrogen peroxide for optimal physiological operations in tissues in vivo should be around 10⁻⁶ molarconcentration. However during adverse conditions, tissue concentrations can reach as high as 10⁻³ Molar which can deactivate enzymes and oxidize amino acids. Tissue concentrations of hydrogen peroxide at 10⁻³ molar have been shown to result in melanocyte apoptosis in the hair follicle and cause DNA damage.

Accordingly, any ingredient or composition topically applied to dermal papilla cells that will modulate hydrogen peroxide concentrations that may result from reactive oxygen species generated from basic metabolism or conditions such as UV damage, physical or psychological stress, pollution, other environmental toxins, and so on, is of value in maximizing the health, color, and integrity of hair, eyelashes and eyebrows.

It has most unexpectedly been discovered that DNA repair enzymes, in particular OGGI type, are most effective in inhibiting or preventing such oxidative damage to dermal papilla cells, particularly oxidative damage that arises from reactive oxygen species.

### SUMMARY OF THE INVENTION

The invention is directed to a composition for topical application to dermal papilla cells associated with keratin fibers from scalp, eyelashes or eyebrows comprising at least one OGG1 DNA repair enzyme.

The invention is also directed to a composition for treating dermal papilla cells associated with keratin fibers from scalp, eyelashes, or eyebrows comprising at least one DNA repair enzyme and inactivated bacterial lysates from Bifido bacterium.

The invention is directed to a method for inhibiting oxidative damage to dermal papilla cells associated with keratin fibers and exposed to reactive oxygen species comprising treating the cells with an effective amount of a DNA repair enzyme.

The invention is also directed to a method for stimulating melanin synthesis in dermal papilla cells associated with keratin fibers and exposed to reactive oxygen species comprising treating the cells with an effective amount of a DNA repair enzyme.

The invention is also directed to a method for reducing apoptosis in dermal papilla cells associated with keratin fibers and exposed to reactive oxygen species by treating the dermal papilla cells with an effective amount of a DNA repair enzyme.

### DESCRIPTION OF FIGURES

FIG. 1 depicts the effects of Roxisomes® on cellular viability at various concentrations.

FIG. 2 depicts the effects of increasing concentrations of Roxisomes® in ameliorating the adverse effects of exposure to hydrogen peroxide.

FIG. 3A depicts histological sections of untreated black hair stained for melanin (Panel 1), proliferation and apoptosis (Panel 2) and tyrosinase (Panel 3).

FIG. 3B depicts histological sections of black hair treated with 0.5% Roxisomes® and stained for melanin (Panel 1), proliferation and apoptosis (Panel 2) and tyrosinase (Panel 3).

FIG. 4C depicts sections of untreated gray hair stained for melanin (Panel 1), proliferation and apoptosis (Panel 2), and tyrosinase (Panel 3).

FIG. 4D depicts sections of gray hair treated with 0.5% Roxisomes® and stained for melanin (Panel 1), proliferation and apoptosis (Panel 2), and tyrosinase (Panel 3).

### I. THE COMPOSITIONS OF THE INVENTION

The invention is directed to compositions for topical application to dermal papilla cells associated with keratin fibers from scalp, eyelashes or eyebrows comprising at least one DNA repair enzyme which is preferably OGG1. The term “associated with keratin fibers” means that the dermal papilla cells are for development of hair on the scalp, eyelashes, or eyebrows.

The composition can be in the form of an emulsion, solution, suspension, or anhydrous preparation. The composition may be a rinse off composition, that is, it is applied to treat the desired area, then rinsed off. The composition may also be a leave on composition, that is, a composition that applied to the surface and left on for the normal period of time associated with the composition. The composition may also be in the form of a shampoo, conditioner, scalp treatment composition, eyelash treatment composition, mascara, or brow treatment preparation.

The composition comprises at least one DNA repair enzyme as further described herein.

A. DNA Repair Enzymes

The DNA repair enzymes used in the invention may be present in the composition in amounts ranging from about 0.00001 to about 5%, preferably from about 0.00005 to about 3%, more preferably from about 0.0001 to about 1%. All percentages mentioned herein are by weight of the total composition.

DNA repair enzymes as disclosed in U.S. Pat. Nos. 5,077,211; 5,190,762; 5,272,079; and 5,296,231, all of which are hereby incorporated by reference in their entirety, are suitable for use in the compositions and method of the invention. One example of such a DNA repair enzyme may be purchased from AGI/Dermatics under the trade name Roxisomes®, and has the INCI name Arabidopsis thaliana extract. It may be present alone or in admixture with lecithin and water or in the form of liposomes. More specifically, this extract may be in the form of a composition containing 0.001 to 2.0%, preferably 0.01 to 1.0, most preferably 0.1 to 0.75%, or specifically 0.5% Arabidopsis thaliana extract; 0.001 to 2.0%, preferably 0.01 to 1.0, most preferably 0.1 to 0.75%, or specifically 0.5% lecithin; and 99% water. This DNA repair enzyme is known to be effective in repairing 8-oxo-diGuanine (OGG1) base mutation damage and is referred to as an OGG1 DNA repair enzyme.

Another type of DNA repair enzyme that may be used is one that is known to be effective in repairing 06-methyl guanine base mutation damage. It is sold by AGI/Dermatics under the tradename Adasomes®, and has the INCI name Lactobacillus ferment, which may be added to the composition of the invention by itself or in admixture with lecithin and water.

Another type of DNA repair enzyme that may be used is one that is known to be effective in repairing T-T dimers. The enzymes are present in mixtures of biological or botanical materials. Examples of such ingredients are sold by AGI/Dermatics under the tradenames Ultrasomes® or Photosomes®. Ultrasomes® comprises a mixture of Micrococcus lysate (an end product of the controlled lysis of various species of micrococcus), lecithin, and water. Photosomes® comprises a mixture of plankton extract (which is the extract of marine biomass which includes one or more of the following organisms: thalassoplankton, green micro-algae, diatoms, greenish-blue and nitrogen-fixing seaweed), water, and lecithin.

Another type of DNA repair enzyme may be a component of various inactivated bacterial lysates from Bifido bacterium, such as Bifida lysate or Bifida ferment lysate, the latter a lysate from Bifido bacteria which contains the metabolic products and cytoplasmic fractions when Bifido bacteria are cultured, inactivated and then disintegrated. This material has the INCI name Bifida Ferment Lysate. Such bacterial lysates are disclosed in U.S. Pat. No. 4,464,362, which is hereby incorporated by reference in its entirety.

Other suitable DNA repair enzymes include Endonuclease V, which may be produced by the denV gene of the bacteriophage T4. Also suitable are T4 endonuclease; O⁶-methylguanine-DNA methyltransferases; photolyases such as uracil- and hypoxanthine-DNA glycosylases; apyrimidinic/apurinic endonucleases; DNA exonucleases, damaged-bases glycosylases (e.g., 3-methyladenine-DNA glycosylase); correndonucleases either alone or in complexes (e.g., E. coli uvrA/uvrB/uvrC endonuclease complex); APEX nuclease, which is a multi-functional DNA repair enzyme often referred to as “APE”; dihydrofolate reductase; terminal transferase; topoisomerase; O⁶ benzyl guanine; DNA glycosylases.

Other types of suitable DNA repair enzymes may be categorized by the type of repair facilitated and include BER (base excision repair) or BER factor enzymes such as uracil-DNA glycosylase (UNG); single strand selective monofunctional uracil DNA glycosylase (SMUG1); 3,N(4)-ethenocytosine glycosylase (MBD4); thymine DNA-glycosylase (TDG); A/G-specific adenine DNA glycosylase (MUTYH); 8-oxoguanine DNA glycosylase (OGG1); endonuclease III-like (NTHL1); 3-methyladenine DNA glycosidase (MPG); DNA glycosylase/AP lyase (NEIL1 or 2); AP endonuclease (APEX 1 and 2), DNA ligase (LIG3), ligase accessory factor (XRCC1); DNA 5′-kinase/3′-phosphatase (PNKP); ADP-ribosyltransferase (PARP1 or 2).

Another category of DNA repair enzymes includes those that are believed to directly reverse damage such as O⁶-MeG alkyl transferase (MGMT); 1-meA dioxygenase (ALKBH2 or ALKBH3).

Yet another category of enzymes operable to repair DNA/protein crosslinks includes Tyr-DNA phosphodiesterase (TDP1).

Also suitable are MMR (mismatch exision repair) DNA repair enzymes such as MutS protein homolog (MSH2); mismatch repair protein (MSH3); mutS homolog 4 (MSH4); MutS homolog 5 (MSH5); or G/T mismatch-binding protein (MSH6); DNA mismatch repair protein (PMS1, PMS2, MLH1, MLH3); Postmeiotic segregation increased 2-like protein (PMS2L3); or postmeiotic segregation increased 2-like 4 pseudogene (PMS2L4).

Also suitable are DNA repair enzymes are those known as nucleotide excision repair (NER) enzymes and include those such as Xeroderma pigmentosum group C-complementing protein (XPC); RAD23 (S. cerevisiae) homolog (RAD23B); caltractin isoform (CETN2); RFA Protein 1, 2, of 3 (RPA1, 2, or 3); 3′ to 5′ DNA helicase (ERCC3); 5′ to 3′ DNA helicase (ERCC2); basic transcription factor (GTF2H1, GTF2H2, GTF2H3, GTF2H4, GTF2H5); CDK activating kinase (CDK7, CCNH); cyclin G1-interacting protein (MNAT1); DNA excision repair protein ERCC-51; excision repair cross-complementing 1 (ERCC1); DNA ligase 1 (LIG1); ATP-dependent helicase (ERCC6); and the like.

Also suitable may be DNA repair enzymes in the category that facilitate homologous recombination and include, but are not limited to DNA repair protein RAD51 homolog (RAD51, RAD51L1, RAD51B etc.); DNA repair protein XRCC2; DNA repair protein XRCC3; DNA repair protein RAD52; ATPase (RAD50); 3′ exonuclease (MRE11A); and so on.

DNA repair enzymes that are DNA polymerases are also suitable and include DNA polymerase beta subunit (POLB); DNA polymerase gamma (POLG); DNA polymerase subunit delta (POLD1); DNA polymerase II subunit A (POLE); DNA polymerase delta auxiliary protein (PCNA); DNA polymerase zeta (POLZ); MAD2 homolog ((REV7); DNA polymerase eta (POLH): DNA polymerase kappa (POLK): and the like.

Various types of DNA repair enzymes that are often referred to as “editing and processing nucleases” include 3′-nuclease; 3′-exonuclease; 5′-exonuclease; endonuclease; and the like.

Other examples of DNA repair enzymes include DNA helicases including such as ATP DNA helicase and so on.

The DNA repair enzymes may be present as components of botanical extracts, bacterial lysates, biological materials, and the like. For example, botanical extracts may contain DNA repair enzymes.

The compositions of the invention may contain one or more DNA repair enzymes. Most preferred is where the composition contains an OGGI DNA repair enzyme, more particularly, a DNA repair enzyme referred to as Roxisomes® which has the INCI name Arabidopsis thaliana extract. This ingredient composition is a mixture of about 0.5 parts of Arabidopsis thaliana extract, 0.5 parts lecithin, and the remainder water. In another preferred embodiment, the composition contains a mixture of DNA repair enzymes, in particular, a mixture of Arabidopsis thaliana extract and inactivated bacterial lysates from Bifido bacterium.

B. Other Ingredients

The composition may contain one or more additional ingredients including but not limited to those set forth herein.

1. Oils

The composition of the invention may contain one or more oils. Suitable oils include silicones, esters, vegetable oils, synthetic oils, examples of which are set forth herein. The oils may be volatile or nonvolatile, and are preferably in the form of a pourable liquid at room temperature. The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C.

Suitable volatile oils generally have a viscosity ranging from about 0.5 to 5 centistokes 25° C. and include linear, cyclic, or branched silicones, paraffinic hydrocarbons, or mixtures thereof. Examples include cyclic silicones having the general formula:

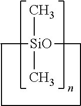

where n=3-6, preferably 4, 5, or 6.

Examples of linear volatile silicones, for example, those having the general formula:

(CH₃)₃Si—O—[Si(CH₃)₂—O]ₙ—Si(CH₃)₃

where n=0, 1, 2, 3, 4, or 5, preferably 0, 1, 2, 3, or 4.

Examples of branched volatile silicones include alkyl trimethicones such as methyl trimethicone, a branched volatile silicone having the general formula:

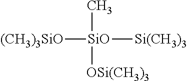

Methyl trimethicone may be purchased from Shin-Etsu Silicones under the tradename TMF-1.5, having a viscosity of 1.5 centistokes at 25° C.

Cyclic and linear volatile silicones are available from various commercial sources including Dow Corning Corporation and General Electric. The Dow Corning linear volatile silicones are sold under the tradenames Dow Corning 244, 245, 344, and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference.

Preferred volatile paraffinic hydrocarbons have a molecular weight of 70-225, preferably 160 to 190 and a boiling point range of 30 to 320, preferably 60 to 260° C., and a viscosity of less than about 10 cst. at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable.

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to esters, hydrocarbons, or silicones.

Suitable esters may include one or more of monoesters, diesters, or triesters. Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described in the C.T.F.A. Cosmetic Ingredient Dictionary and Handbook, Eleventh Edition, 2006, under the classification of “Esters”, the text of which is hereby incorporated by reference in its entirety.

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

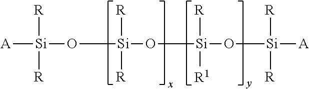

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814.

2. Pigments or Powders

The compositions of the invention may contain particulate materials in the form of pigments, inert particulates (e.g. powders) or mixtures thereof. If present, suggested ranges are from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

The particulate matter may be colored or non-colored (for example white) non-pigmented powders. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

3. Surfactants

The composition may contain one or more surfactants, especially if in the emulsion form. However, such surfactants may be used if the compositions are anhydrous also, and will assist in dispersing ingredients that have polarity, for example pigments. Such surfactants may be silicone or organic based. The surfactants will aid in the formation of stable emulsions of either the water-in-oil or oil-in-water form. If present, the surfactant may range from about 0.001 to 30%, preferably from about 0.005 to 25%, more preferably from about 0.1 to 20% by weight of the total composition.

Suitable surfactants include silicone and/or organic surfactants. Examples of silicone surfactants are polyorganosiloxane polymers that have amphiphilic properties, for example contain hydrophilic radicals and lipophilic radicals. These silicone surfactants may be liquids or solids at room temperature.

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One more specific type of suitable silicone surfactant has the general formula:

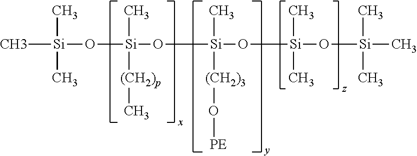

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

Another type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer, which provides excellent aesthetics due to its elastomeric backbone, but also surfactancy properties.

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

4. Structuring Agents

The composition may contain one or more structuring agents. The agents may structure, or increase viscosity, in either the water or oil phases or both. If present, the structuring agent may range from about 0.1 to 30%, preferably from about 0.5 to 25%, more preferably from about 1 to 20%.

Silicone elastomers are one example of suitable structuring agents that may be used in the composition of the invention. In general they are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Corning's DC9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crossoplymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety.

Also suitable for use as structuring agents are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges, e.g. 550,000; 925,000; 3.5 million.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the INCI name dimethicone.

Another type of structuring agent includes silicone waxes that are typically referred to as alkyl silicone waxes which are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, or behenyl dimethicone, behenoxy dimethicone.

Also suitable as structuring agent may be one or more natural or synthetic waxes such as animal, vegetable, or mineral waxes. Preferably such waxes will have a higher melting point such as from about 50 to 150° C., more preferably from about 65 to 100° C. Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.

Another type of structuring agent that may be used in the compositions are silicas, silicates, silica silylate, and alkali metal or alkaline earth metal derivatives thereof. These silicas and silicates are generally found in the particulate form and include silica, silica silylate, magnesium aluminum silicate, and the like.

5. Sunscreens

It may also be desirable to include one or more sunscreens in the compositions of the invention. Such sunscreens include chemical UVA or UVB sunscreens or physical sunscreens in the particulate form.

The term “UVA sunscreen” means a chemical compound that blocks UV radiation in the wavelength range of about 320 to 400 nm. Preferred UVA sunscreens are dibenzoylmethane compounds having the general formula

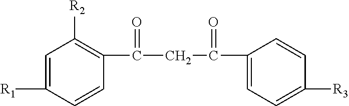

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

Other types of UVA sunscreens include dicamphor sulfonic acid derivatives, such as ecamsule, a sunscreen sold under the trade name Mexoryl™, which is terephthalylidene dicamphor sulfonic acid, having the formula:

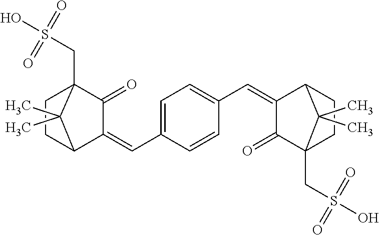 [SMILES: CC1(C)C2CCC1(CS(=O)(=O)O)C(=O)/C2=C\c1ccc(/C=C2\C(=O)C3(CS(=O)(=O)O)CCC2C3(C)C)cc1]

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

The term “UVB sunscreen” means a compound that blocks UV radiation in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 110% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

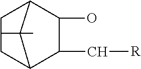

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

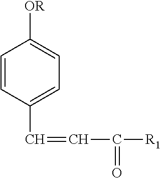

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the general formula:

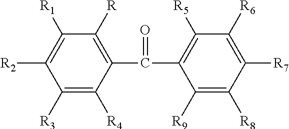

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

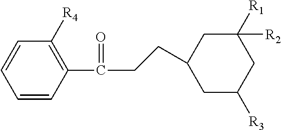

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

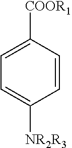

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula:

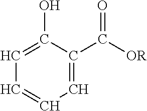

wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

In the case where the composition is applied to keratin fibers that are scalp hair, including one or more chemical sunscreens may be very helpful in increasing the effectiveness of the DNA repair enzymes.

The composition may contain a variety of other ingredients including but not limited to preservatives, vitamins, anti-oxidants, and the like.

### II. THE FORMS OF THE COMPOSITION

The compositions of the invention may be in the form of hair treatment compositions (such as shampoo, conditioner, scalp treatments, etc.) or products for application to lashes or brows such as lash treatments, mascara, brow treatments, or brow color. In addition to containing the DNA repair enzyme in the amounts set forth herein, more preferred embodiments of the compositions are set forth below.

One suitable shampoo composition may comprise:

10-95% water,
0.1-20% of anionic, zwitterionic, amphoteric, betaine or cationic surfactants (including but not limited to babassuaminopropyl betaine, sodium methyl cocoyl taurate, sodium cocoyl isethionate, sodium lauroamphoacetate, disodium laureth sulfosuccinate, cocamide MIPA, and the like; and optionally
0.1-20% conditioning ingredients such as oils including those mentioned herein.

Another suitable shampoo composition may comprise:

50-99% water,
0.1-20% of a betaine surfactant,
0.1-10% of a cationic surfactant,
0.1-3% creatine.

Another suitable shampoo composition may comprise:

50-99% water,
0.1-20% of a babassuamidopropyl betaine,
0.1-5% sodium cocoyl taurate,
0.1-5% of a fatty alcohol,
0.1-3% of cetyl trimethylammonium chloride.

A suitable conditioner composition may comprise:

65-95% water,
0.1-20% of one or more fatty alcohols (e.g. cetyl or cetearyl alcohol),
0.1-30% of a cationic conditioning agent.

Another suitable conditioner composition may comprise:

65-95% water,
0.1-20% cationic conditioning agent,
0.1-5% creatine.

Another suitable hair conditioner composition may comprise:

65-95% water
0.1-20% cationic conditioning agent,
0.1-10% botanical extracts, and
0.1-5% of an ingredient selected from the group consisting of carnitine, acetyl carnitine, creatine, adenosine phosphate and mixtures thereof.

Another suitable hair conditioner may comprise

65-95% water,
0.1-20% cationic conditioning agent,
0.1-10% fatty alcohol,
0.1-5% of an ingredient selected from the group consisting of carnitine, acetyl carnitine, creatine, adenosine phosphate and mixtures thereof.

Suitable scalp treatment compositions may be anhydrous or aqueous based. If anhydrous, the may comprise oils and other ingredients. If aqueous based, the composition may be in the emulsion or solution form. Suitable emulsions may contain from 10-95% water, 0.1-20% oil and other ingredients. In the event the composition is in the solution form, the composition may contain 80-99% water and other ingredients.

Suitable scalp treatment compositions may comprise:

80-99% water,
0.1-5% of an ingredient selected from the group consisting of creatine, adenosine phosphate, carnitine, acetyl carnitine, and mixtures thereof,
0.1-10% yeast extract,
0.1-5% botanical extracts,
0.1-5% of an ingredient selected from the group consisting of glucosamine, N-acetyl glucosamine and mixtures thereof.

Another suitable scalp treatment composition may comprise:

70-99% water,
0.1-5% botanical extracts,
0.1-10% oil.

Suitable lash treatment compositions may comprise:

75-99% water,
0.1-10% humectants,
0.1-5% of an ingredient selected from the group consisting of creatine, adenosine phosphate, carnitine, acetyl carnitine, and mixtures thereof,
0.1-3% arginine.

Another suitable lash or brow treatment compositions may comprise:

65-99% water,
0.1-5% of an ingredient selected from the group consisting of creatine, adenosine phosphate, carnitine, acetyl carnitine, NADH and mixtures thereof,

Suitable lash color compositions may comprise:

1-30% film forming polymer (such as dimethicone silylate),
0.1-5% dimethicone gum,
0.1-25% pigments.

Another suitable lash color composition may comprise:

5-75% water,
1-30% film forming polymer,
0.1-10% surfactant, and
0.1-25% pigments.

### III. THE METHOD

The invention is also directed to a method for inhibiting oxidative damage to dermal papilla cells associated with keratin fibers and exposed to reactive oxygen species comprising treating the cells with an effective amount of a DNA repair enzyme.

The dermal papilla cells may be associated with keratin fibers from scalp hair, eyelashes, or eyebrows. In the method of the invention the dermal papilla cells are exposed to reactive oxygen species that may arise as a result of environmental conditions such as pollution, environmental toxins, or even extreme weather conditions. The reactive oxygen species may also originate from physical or psychological stress, or unhealthy body conditions that cause higher than normal concentration of hydrogen peroxide in tissues.

The invention is also directed to a method for stimulating melanin synthesis in dermal papilla cells associated with keratin fibers and exposed to reactive oxygen species comprising treating the cells with an effective amount of a DNA repair enzyme. The same compositions

The invention is also directed to a method for reducing apoptosis in dermal papilla cells associated with keratin fibers and exposed to reactive oxygen species by treating the dermal papilla cells with an effective amount of a DNA repair enzyme.

The dermal papilla cells may be treated using the DNA repair enzyme alone or in solution or suspension in water or other solvents. Alternatively, the compositions mentioned herein may also be used to treat the dermal papilla cells with the type of composition depending on the location of the dermal papilla cells. For example, dermal papilla cells on the scalp may be treated with scalp treatment compositions, shampoos, conditioners, or even styling agents so long as the compositions applied to the keratin fibers reach the dermal papilla cells. The treatments may vary from one two times per day up to once or twice a week or month. The compositions may be applied as leave on or rinse off compositions.

In the case where it is desired to treat dermal papilla cells associated with eyebrows or eyelashes, the compositions used to treat may be in the form of lash or brow treatments or mascaras, for which examples of such compositions are set forth herein.

The compositions and methods of the invention are shown to reduce oxidative damage due to reactive oxygen species in such dermal papilla cells, and in addition, to reduce apoptosis and stimulate melanin synthesis.

The invention will be further described in connection with the following examples which are set forth for the purposes of illustration only.

### Example 1

An in vitro dermal papilla assay was used to test the effects of Roxisomes® at four different concentrations on cellular viability and effectiveness in inhibiting hydrogen peroxide initiated cytotoxicity.

### Materials:

tissue culture treated plates, 96 well, Corning/Costar

human follicle dermal papilla cells—PromoCell (“HFDPC”)

follicle dermal papilla cell growth medium (“Growth Medium”), PromoCell

Supplement mix, PromoCell

Hepes BSS, PromoCell

Penicillin streptomycin, Invitrogen

Trypsin/EDTA, PromoCell

0.05% Trypsin inhibitor, 0.1% BSA, PromoCell

Phosphate buffered saline (“PBS”), Sigma

Testosterone, Sigma

Hydrogen peroxide solution, Sigma

DMSO, Sigma

MTT Cell Proliferation Assay, ATCC

Test samples were combined with Growth Media, mixed well and filtered.

In order to determine the appropriate experimental concentration of hydrogen peroxide for the test, HFDPC were treated with hydrogen peroxide at the following concentrations (% v/v): 0.1, 0.05, 0.025, 0.0125, 0.063, 0.0031, 0.0016, 0.0008, 0.0004 and 0 overnight. Performance of the MTT Cell Proliferation Assay (ATCC, Cat. No. 30.1010K) according to kit directions showed a clear line of live or dead cells between 0.0031 and 0.0063% (0.407 and 0.084, respectively). After an additional titer was run at 0.0031 and 0.0063%, it was decided that 0.005% was the appropriate experimental concentration of hydrogen peroxide for performing this test.

One vial of HFDPC was thawed and put into a T-75 flask. Cells were allowed to grow until confluent. The cells were trypsinized and resuspending in 3 ml of complete media which is the growth media+Streptomycin provided by PromoCell in the kit. A cell count was performed by adding 100 μl of cell suspension to 80 μl of PBS and 20 μl of Trypan Blue. The cells were resuspended to a concentration of 40,000 cells/ml. 100 μl of cell suspension to each well of three 96 well plates (4,000 cells/well). All plates were returned to 37° C./5% CO₂ incubator for 24 hours.

The test samples were diluted to the appropriate concentrations in Growth Media. 100 μl of each test sample dilution was added to the cells in the wells in triplicate. Then 100 μl of Growth Media was added to the media and hydrogen peroxide wells with the Media well being the vehicle control and the hydrogen peroxide the positive control. Three replicate 96 well plates were prepared.

Plates were then incubated for 48 hours in the presence of the test samples at 37° C./5% CO₂. Then 2 μl of Growth Media was added to the Media control wells. The plates were incubated overnight at 37° C./5% CO₂.

The MTT kit was removed from refrigerated storage and allowed to equilibrate to room temperature. Ten μl of MTT Reagent was added to all wells containing Growth Media. The plates were swirled to ensure mixing. The plates were returned to 37° C./5% CO₂ incubator for 2-3 hours or until all cells clearly showed purple precipitate, but no longer than 4 hours. Then 100 μl of MTT detergent (from kit) was added to all wells and the plate swirled to ensure mixing, taking care not to introduce bubbles in the wells. The plates were covered and stored at room temperature for 4 hours or overnight. The plates were then read at 570 nm.

Data from the readings was analyzed using a GraphPad InStat version 3.00 for Windows 95. A one way ANOVA with Dunnet's post test was performed to determine significance.

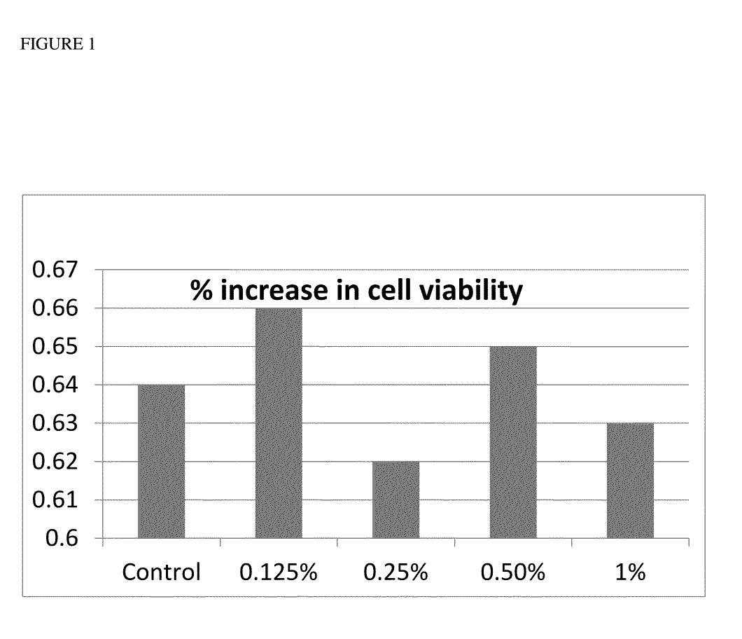

The results showed that the human follicle dermal papilla cells treated with Roxisomes® at concentrations of 0.125, 0.25, 0.5, and 1% concentration showed no statistically significant change in cellular viability when compared with untreated control cells indicating that Roxisomes® were not cytotoxic to cells at any concentration. The results are further illustrated in FIG. 1 and in the Table below.

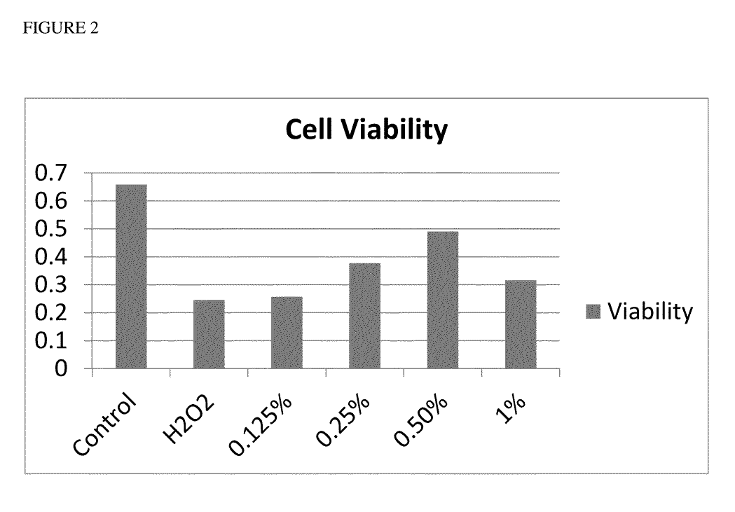

For HFDPC's treated with Roxisomes® for 24 hours and then exposed to hydrogen peroxide for 24 hours showed a dose dependent increase in cell viability up to 0.5% Roxisomes® with a significant increase in viability at 0.5% Roxisomes® compared to untreated control. The results are set forth in FIG. 2 and the Table below, and show that increasing concentrations of Roxisomes® are effective in ameliorating the adverse effects of exposure to hydrogen peroxide up to 0.5% concentration. At a concentration of 1% Roxisomes® the effectiveness decreases slightly indicating that there may be a cytotoxic effect at Roxisome® concentrations at 1%.

| | Cell Viability | | | | |
|---|---|---|---|---|---|
| | | Concentration | | | |
| Test Materials | | 0.125% | 0.25% | 0.50% | 1% |
| Untreated Cells | 0.64 | — | — | — | — |
| Cells treated with | 0.24 | — | — | — | — |
| 0.005% H₂O₂ | | | | | |
| Cells treated with | — | 0.66 | 0.62 | 0.65 | 0.63 |
| Roxisomes ® only | | | | | |
| Cells treated with | — | 0.26 | 0.38 | 0.49 | 0.31 |
| H₂O₂ and Roxisomes ® | | | | | |

The above results show that in all cases treatment of hydrogen peroxide exposed cells with Roxisomes® at all concentrations ameliorates the cytotoxic effect of hydrogen peroxide on cells with the ameliorative effect decreasing slightly at a concentration of 1% Roxisomes®.

### Example 2

Hair follicles from the occipital region of hair transplant candidates were supplied by a local plastic surgeon. Individual hairs were placed into separate wells of 6-well cell culture plates (Corning/Costar, #3506) containing 2 mL of William's E Medium (Sigma, #W4128) supplemented with 2 mM L-glutamine Acros Organics, #119951000), 10 μg/mL insulin (Sigma, #I0908), 10 ng/mL hydrocortisone (Sigma, #H0396), and 100 units/mL pencillin/100 μg/mL streptomycin Cellgro, #30-001-CI) to maintain the hair follicles (as described by Philpott et al. 1994). Hairs were stabilized in media over the weekend at 37° C./5% CO₂.

On day 0, media was aspirated, and 2 mL of each treatment was added to wells (6 hairs per treatment), as follows:

1) Media, untreated control

2) 0.5% (v/v) Roxisomes® in media

3) 1% (v/v) Roxisomes® in media

4) Minoxidil (Sigma, M4145) in media, positive control

Hairs were incubated at 37° C./5% CO₂ during the course of the experiment. Treatments were replaced every 2 days. The experiment ran for 9 days, after which time the hair was washed 2 times with 2 mL of phosphate buffered saline (PBS) for 5 min, and then fixed in 5% formalin (in PBS) overnight at 4° C. The following day, the hairs were washed 2 times with 2 mL of PBS for 5 min and then stored in 70% ethanol at 4° C.

Histological techniques facilitate the evaluation of the effects of topical treatments at a microscopic level. In this study, we used the following histological stains to visualize the effects of Roxisomes® on cell viability and melanin production:

1) Fontana-Masson silver stating is used to reveal the presence of melanin in tissue sections as a result of the melanin granules reducing silver nitrate to metallic silver, a histochemical reaction that reveals accumulations of black material wherever melanin is located (Patterson, J. W., Cutis, Vol. 74, 293-209 2004).

2) Tyrosinase is an enzyme involved in the initial stages of melanin biosynthesis in melanocytes; tyrosinase staining is an indicator for melanin synthesis (Clarkson et al., Journal of Clinical Pathology, Vol. 54, 196-200. 2001).

3) Ki67 staining detects cellular proliferation (Scholzen and Gerdes 2000), and indication that cells are dividing and viable. Cleaved Caspase-3 is an antibody that detects cells in apoptosis (Gown and Willingham 2002). Apoptosis in turn is an indicator of cell death.

Histological sectioning and staining of hairs was performed using the following protocols.

### Fontana Masson Silver Staining

Tissue sections, 3 uM, were cut and mounted to slides. Slides were deparaffinized and hydrated with distilled water

Slides were placed in silver nitrate solution (Poly-Scientific, Bayshore, N.Y. #2181S) at 56° C.

Slides were then incubated with gold chloride 0.2% aqueous (Poly-scientific, #s201) for 10 minutes and rinsed again in distilled water

Slides were then incubated with sodium thiosulfate 5% (Fisher Scientific, Pittsburgh, Pa. #S474) for 5 minutes, then rinsed in distilled water

Slides were then incubated with nuclear fast red (Poly-scientific, #s248) counter stain for 5 minutes, then rinsed well with distilled water

Slides were dehydrated in graded alcohols and cover slipped

### Tyrosinase Staining

3 μm tissue sections were cut and mounted to slides. Slides were de-paraffinized and hydrated with distilled water
Antigen retrieval for 1 hr at 90° C. in Trilogy solution (Cell Marque, Rocklin, Calif., #92p-10) was performed on the slides
Slides were rinsed for 10 minutes in Tris buffered saline and Tween 20 (TBS) (Thermo Scientific, Waltham, Mass., #TA-999-TT).
Slides were then treated with protein block for 10 minutes (Biocare Medical, Concord, Calif., #BS966M) and then rinsed in TBS for 5 minutes
Slides then subjected to primary incubation with anti-tyrosinase antibody (monoclonal mouse antibody, clone T311, Cell Marque #344M-98) for 1 hr at room temperature then rinsed in TBS for 5 minutes
Slides treated with alkaline phosphatase probe for 20 min (Biocare Medical #UP536L) and rinsed in TBS for 5 minutes
Alkaline phosphatase polymer was applied to slides for 30 min (Biocare Medical #MRAP536L), after which they were rinsed in TBS for 5 minutes
Chromogen Red was applied to slides for 15 minutes (Biocare Medical #FR805CHC) followed by rinsing in distilled water and counterstaining with 2 dips in hematoxylin
Slides were dehydrated in graded alcohols, cleared in xylene, and cover slipped.

### Ki67 and Caspase-3 Double Staining

Cut 3-μm tissue sections, and mounted to slides; deparaffinized and hydrated slides to distilled water
Slides subjected to antigen retrieval for 1 hr at 90° C. in Trilogy solution (Cell Marque, Rocklin, Calif., #92p-10) then rinsed for 10 minutes in Tris buffered saline and Tween 20 (TBS) (Thermo Scientific, Waltham, Mass., #TA-999-TT).
The slides were blocked with peroxidazed-1 (Biocare Medical, Concord, Calif., #PX968) for 5 minutes then rinsed in TBS
Slides were blocked with background punisher for 5 minutes (Biocare Medical #BP974) followed by rinsing in TBS
Slides were incubated with Ki67+Caspase-3 antibody (Biocare Medical #PPM240DS) for 1 hr at room temperature followed by rinsing in TBS for 2 minutes
Slides were incubated with Mach 2 Double Stain 2 (Biocare Medical #MRCT525) for 25 minutes, then rinsed in TBS
Slides then incubated with Warp Red Chromogen (Biocare Medical #WR806) for 8 minutes followed by rinsing in PBS (Polyscientific #S2261).
Slides were then incubated with Vina Green Chromogen (Biocare Medical #BRR807) for 10 minutes and rinsed in distilled water
Slides dipped in hematoxylin (Cancer Diagnostics, Morrisville, N.C., #CM3951) and rinsed with distilled water, followed by two dips in TBS
Slides rinsed in distilled water and air dried, then dipped in xylene and coverslipped
   Caspase-3 stains red; Ki67 stains green.

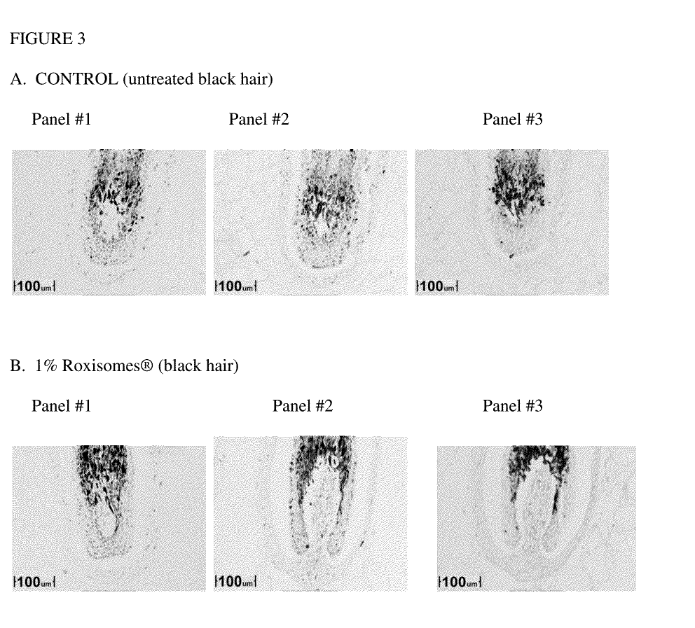

FIG. 3A shows histological sections of untreated black hair stained for melanin (Fontana Masson, Panel #1), cellular proliferation (K67, green) and apoptosis (Caspase-3, red), Panel #2), and tyrosinase (Panel #3).

In FIG. 3A: Panel #1 shows that histological sections of untreated black hair shows the presence of significant amounts of melanin. Panel #2 shows that sections of untreated black hair show appreciable amounts of cellular proliferation, meaning that the cells are dividing; and minimal apoptosis, which indicates cell death. Panel #3 shows presence of significant amount of tyrosinase, a precursor to melanin.

In FIG. 3B: histological sections of black hair treated with 1% Roxisomes®. Panel #1 shows that the presence of melanin in the hair appears to have increased significantly compared to untreated hair in FIG. 3A, Panel #1, thus indicating the positive effect of Roxisomes® in stimulating melanin synthesis (and color) in hair. Panel #2 also shows an increase in cellular proliferation after treatment with Roxisomes®, and an apparent reduction in apoptosis. Panel #3 shows an increase in tyrosinase, a precursor of melanin, in black hair sections treated with Roxisomes®.

Accordingly, treatment of black hair sections with Roxisomes® is histologically shown to improve hair color (melanin), hair health and viability (proliferation), reduce cell death (apoptosis), and facilitate melanin production in hair (tyrosinase).

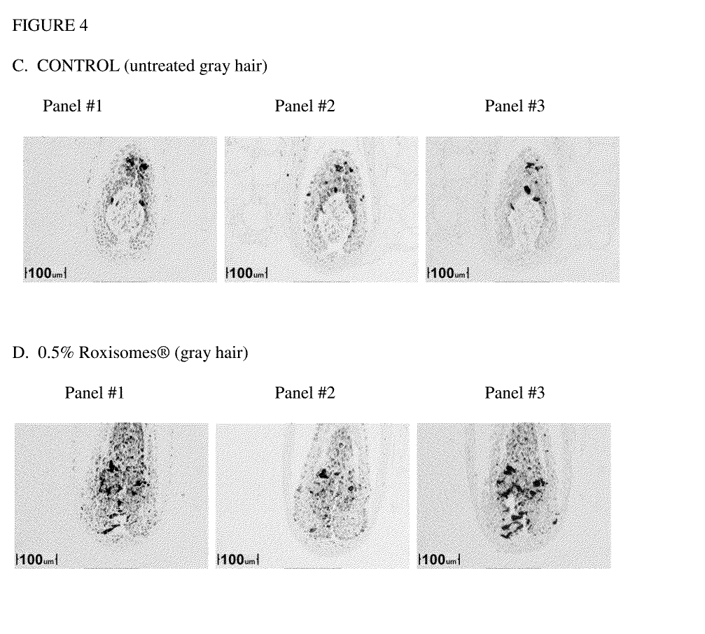

FIG. 4C shows histological sections of untreated gray hair stained for melanin (Fontana Masson, Panel #1), cellular proliferation (K67, green) and apoptosis (Caspase-3, red), Panel #2), and tyrosinase (Panel #3). FIG. 4C, Panel #1 shows gray hair had some melanin present but considerably less than what is seen with untreated black hair. FIG. 4C, Panel #2 shows some moderate amount of cellular proliferation and very little apoptosis. FIG. 4C, Panel #3 shows small amounts of tyrosinase.

FIG. 4D shows histological sections of gray hair treated with 0.5% Roxisomes®: stained for melanin (Fontana Masson, Panel #1), cellular proliferation (K67, green) and apoptosis (Caspase-3, red), Panel #2), and tyrosinase (Panel #3).

Gray hair sections treated with Roxisomes® show a very significant increase in melanin. Panel #2 shows some proliferation and a small amount of apoptosis. Panel #3 shows a definite increase in tyrosinase, a melanin precursor.

Thus, it is seen that treatment of gray hair sections with 0.5% Roxisomes® improves hair color (melanin), improves cellular proliferation (health and viability) and increases presence of tyrosinase.

In both cases treatment of hair sections with Roxisomes® shows improvement in melanin content of hair, cellular viability and reduced cellular death, and tyrosinase.

### Example 3

A shampoo composition is prepared as follows:

| | Wt % |
|---|---|
| Shampoo | % |
| WATER\AQUA\EAU | QS100 |
| Babassuamidiopropyl Betaine | 10.000 |
| Guar Hydroxypropyltrimonium Chloride | 0.500 |
| Sodium Hydroxide | 0.020 |
| 1,3-propanediol | 1.000 |
| Sodium Methyl Cocoyl Taurate | 15.000 |
| WATER\AQUA\EAU/SODIUM COCOYL | 2.000 |
| ISETHIONATE/SODIUM | |
| LAUROAMPHOACETATE/SODIUM METHYL | |
| COCOYL TAURATE | |
| Disodium Laureth Sulfosuccinate | 5.000 |
| Cocamide MIPA | 5.000 |
| Sodium Cocoyl Isethionate | 4.000 |
| Stearamidopropyl Dimethylamine | 0.500 |
| Salicylic Acid | 0.150 |
| WATER\AQUA\EAU | 2.000 |
| Vitis Vinifera (Grape) Seed Extract | 0.100 |
| Potassium Sorbate | 0.150 |
| CARTHAMUS TINCTORIUS(SAFFLOWER) | 3.000 |
| OLEOSOMES/GLYCERIN/ | |
| WATER\AQUA\EAU | |
| Cetrimonium Chloride | 2.000 |
| Butyl Avocadate | 0.100 |
| ETHYL MACADAMIATE | 0.250 |
| Dipotassium Glycyrrhizate | 0.025 |
| PANAX GINSENG (GINSENG) ROOT EXTRACT | 0.100 |
| Purified Water/Malt Extract/Simethicone/Acetyl | 1.000 |
| Carnitine HCL/Adenosine | |
| Phosphate/Creatine/Phenoxyethanol/Yeast Extract | |
| Hydrolyzed Soy Protein | 0.500 |
| Arabidopsis thaliana extract (Roxisomes ®) | 0.01 |
| Citric Acid | 0.500 |
| Sodium Chloride | 1.000 |
| Fragrance (Parfum) and minors | q.s. |

The composition was prepared by combining the ingredients and mixing well.

### Example 4

| Conditioner | w.t. % |
|---|---|
| WATER\AQUA\EAU | QS100 |
| PANAX GINSENG (GINSENG) ROOT EXTRACT | 0.100 |
| Vitis Vinifera (Grape) Seed Extract | 0.100 |
| Guar Hydroxypropyltrimonium Chloride | 0.350 |
| TAPIOCA STARCH | 0.250 |
| Cetrimonium Chloride | 2.000 |
| Potassium Sorbate | 0.100 |
| Caffeine | 0.050 |
| Behentrimonium Methosulfate (and) Cetearyl Alcohol | 4.500 |
| Cetearyl Alcohol | 2.000 |
| Cetyl Alcohol | 2.000 |
| METHYL SOYATE | 1.500 |
| HYDROGENATED OLIVE OIL | 0.100 |
| UNSAPONIFIABLES/HYDROGENATED | |
| ETHYLHEXYL OLIVATE | |
| ALEURITES MOLUCCANA (KUKUI) SEED OIL | 1.000 |
| PERILLA OCYMOIDES SEED EXTRACT/PUNICA | 0.500 |
| GRANATUM (POMEGRANATE) SEED | |
| OIL/SISYMBRIUM IRIO SEED OIL | |
| ETHYL MACADAMIATE | 0.300 |
| glyceryl caprylate | 0.300 |
| CURCUMA LONGA (TURMERIC) ROOT EXTRACT | 0.050 |
| EMBLICA OFFICINALIS FRUIT EXTRACT | 0.050 |
| Hydrolyzed Soy Protein | 0.500 |
| WATER/DIHYDROXYPROPYL ARGININE HCL | 1.000 |
| Purified Water/Malt Extract/Simethicone/Acetyl | 1.000 |
| Carnitine HCL/Adenosine | |
| Phosphate/Creatine/Phenoxyethanol/Yeast Extract | |
| Arabidopsis thaliana extract (Roxisomes ®) | 0.1 |
| Fragrance (Parfum) and minors | 0.300 |

### Example 5

A scalp treatment serum is prepared as follows:

| Scalp Treatment Serum | w.t. % |
|---|---|
| WATER\AQUA\EAU | QS100 |
| Rosmarinus Officinalis (Rosemary) Leaf Extract | 0.001 |
| Hydroxypropyl Methycellulose | 0.300 |
| Caffeine | 0.050 |
| Adenosine Phosphate | 0.040 |
| Dipotassium Glycyrrhizate | 0.100 |
| PANAX GINSENG (GINSENG) ROOT EXTRACT | 0.100 |
| CAMELLIA SINENSIS (GREEN TEA) LEAF EXTRACT | 0.050 |
| Arginine | 0.100 |
| Glucosamine HCl | 0.550 |
| PEG-40 Hydrogenated Castor Oil | 0.900 |
| ALEURITES MOLUCCANA (KUKUI) SEED OIL | 0.001 |
| Yeast Extract | 1.000 |
| SACCHAROMYCES LYSATE EXTRACT/WATER (AQUA | 0.100 |
| PURIFICATA) PURIFIED/ROSMARINUS OFFICINALIS | |
| (ROSEMARY) LEAF EXTRACT | |
| WATER/CHLORELLA VULGARIS EXTRACT | 0.500 |
| Alcohol Denat. | 69.008 |
| Hydroxypropylcellulose | 0.200 |
| Glycyrrhiza Glabra (Licorice) Extract | 0.100 |
| Salicylic Acid | 0.050 |
| CURCUMA LONGA (TURMERIC) ROOT EXTRACT | 0.050 |
| Tocopheryl Nicotinate | 0.200 |
| Tocopheryl Acetate | 0.200 |
| Centella Asiatica (Hydrocotyl) Extract | 0.100 |
| Arabidopsis thaliana extract (Roxisomes ®) | 0.10 |
| Fragrance (Parfum) | 0.300 |

### Example 6

An eyelash treatment composition is prepared as follows:

| Eyelash Treatment Composition | wt % |
|---|---|
| WATER\AQUA\EAU | QS100 |
| Butylene glycol | 3.00 |
| Hydroxyethylcellulose | 1.15 |
| Glycerin | 1.00 |
| Creatine | 0.50 |
| Adenosine phosphate | 0.50 |
| Phenoxyethanol | 0.40 |
| Hydroxypropylmethyl cellulose | 0.40 |
| Acetyl carnitine HCl | 0.10 |
| Arginine | 0.10 |
| Carbomer | 0.10 |
| Ethylhexylglycerin | 0.10 |
| Disodium NADH | 0.05 |
| Ammonium hydroxide | 0.05 |
| Arabidopsis thaliana extract (Roxisomes ®) | 0.10 |

### Example 7

A mascara composition may be prepared as follows:

| Mascara Composition | wt % |
|---|---|
| Dimethicone silylate/isododecane (40:60) (DC 7-4405) | QS100 |
| Dimethicone gum (GE Silicones SE-30) | 10.00 |
| Trimethylsiloxy silicate | 5.00 |
| Hydrogenated styrene/isoprene copolymer 20 Black 2/polyester- | 2.50 |
| 5/PVP/Laureth-4 (40:31:24.5:4.5) | |
| FD&C Blue No. 1 Aluminum Lake 0.50 | 0.50 |
| Caprylyl glycol/phenoxyethanol/hexylene glycol (55:35:10) | 0.30 |
| Panthenol | 0.10 |
| Panthenyl ethyl ether | 0.10 |
| Arabidopsis thaliana extract (Roxisomes ®) | 0.10 |

The composition is prepared by combining the ingredients and mixing well.

1. A composition for topical application to dermal papilla cells associated with keratin fibers from scalp, eyelashes or eyebrows comprising at least one OGG1 DNA repair enzyme.

2. The composition of claim 1 where the OGG1 DNA repair enzyme is present in an amount ranging from 0.00001 to 5%.

3. The composition of claim 2 further comprising at least one inactivated bacterial lysate of Bifido bacterium.

4. The composition of claim 1 which is a rinse off composition or a leave on composition.

5. The composition of claim 1 which is a shampoo, hair conditioner, scalp treatment composition, lash treatment composition, brow treatment composition, mascara, or brow color.

6. The composition of claim 1 wherein the DNA repair enzyme is Arabidopsis thaliana extract.

7. The invention is directed to a method for inhibiting oxidative damage to dermal papilla cells associated with keratin fibers and exposed to reactive oxygen species comprising treating the cells with an effective amount of a DNA repair enzyme.

8. The method of claim 7 wherein the keratin fibers are scalp hair, eyelashes, or eyebrows.

9. The method of claim 7 wherein the DNA repair enzyme is OGG1.

10. The method of claim 7 wherein the DNA repair enzyme is applied in the form of a rinse off composition or a leave on composition.

11. The method of claim 10 wherein the rinse off composition is a shampoo or hair conditioner.

12. The method of claim 10 wherein the composition is a leave on composition.

13. The method of claim 11 wherein the leave on composition is a hair treatment composition, a lash treatment, a brow treatment, a lash color, or a brow color.

14. A method for stimulating melanin synthesis in dermal papilla cells associated with keratin fibers and exposed to reactive oxygen species comprising treating the cells with an effective amount of a DNA repair enzyme.

15. The method of claim 14 where the DNA repair enzyme is OGG1.

16. The method of claim 15 wherein the DNA repair enzyme is present at 0.00001 to 5% by weight of the total composition.

17. The method of claim 14 wherein the DNA repair enzyme is Arabidopsis thaliana extract.

18. The method of claim 14 wherein the cells are treated with a leave on composition or a rinse off composition.

19. A method for reducing apoptosis in dermal papilla cells associated with keratin fibers and exposed to reactive oxygen species by treating the dermal papilla cells with an effective amount of a DNA repair enzyme.

20. The method of claim 19 wherein the DNA repair enzyme is OGG1.
